(19) United States
(12) Patent Application Publication
(10) Pub. No.: US 2002/0064741 A1
(43) Pub. Date: May 30, 2002

(54) Premixing burner block for partial oxidation processes

(76) Inventors: Bernd Bartenbach, Limburgerhof (DE); Dieter Stapf, Mannheim (DE); Michael Bachtler, Albersweiler (DE); Olaf Scheidsteger, Mannheim (DE); Peter Passler, Ludwigshafen (DE)

Correspondence Address: KEIL & WEINKAUF, 1350 CONNECTICUT AVENUE, N.W., WASHINGTON, DC, 20036, US

(21) Appl. No.: 09/929,074

(22) Filed: Aug. 15, 2001

(30) Foreign Application Priority Data
Aug. 25, 2000 (DE) 10041739.6

(51) Int. Cl. C07C 11/24; F23M 3/02; F23C 5/00; F23Q 9/00; C07C 2/78; C07C 4/06; C07C 11/00; F23D 5/00

(52) U.S. Cl. 431/8; 431/278; 431/284; 431/201

(57) ABSTRACT

The present application relates to a burner block (1) comprising a base (2), a plurality of hydrocarbon channels (3) for the passage of hydrocarbon/air mixtures used as reactants, and at least one oxygen channel (4) for the passage of air or an air/oxygen mixture. An ignition burner (5) is optionally present. The burner block allows the combustion of hydrocarbons for the preparation of acetylene at temperatures of <1400° C.

The present invention relates to a novel burner block which can be employed in partial oxidation processes, preferably the combined preparation of acetylene and synthesis gas from methane, liquid gas, naphtha or similar starting substances. The burner block is particularly suitable for use in partial oxidation processes carried out in newly developed low-temperature procedures.

Numerous processes for the uncatalyzed preparation of acetylene are based on the pyrolysis or partial oxidation of hydrocarbons. With natural gas, various petroleum fractions (for example including naphtha) and even residue oils (submerged-flame processes), a broad range of starting substances are used. Thermodynamic and kinetic parameters always have a crucial effect on the choice of reaction conditions in pyrolytic or oxidative processes for the preparation of acetylene. Important prerequisites of corresponding processes in classical methods are rapid energy supply at a reaction temperature of greater than 1400° C., extremely short residence times of the starting materials or reaction products of from 10⁻² to 10⁻³ seconds, low partial pressure of the acetylene and rapid quenching of the gases formed. On pyrolysis and partial oxidation, acetylene is obtained in the form of a gas mixture, the so-called cracking gas. The cracking gas normally comprises from about 5 to 20% by volume of acetylene. The acetylene is extracted from the cracking gas by, for example, N-methylpyrrolidone, dimethylformamide, kerosene, methanol or acetone and subsequently purified further.

The individual processes in which acetylene is prepared differ in particular with respect to the generation of the high reaction temperatures. The provision and transfer of the thermal energy play a crucial role here.

The Sachsse-Bartholomé process of the applicant is a so-called autothermal cracking process and suitable for starting materials, such as methane, liquid gas or petroleum ether. The majority of the plants constructed worldwide are based on natural gas as starting material; only a few use naphtha as raw material. In the industrial process, methane and oxygen, for example, are preheated separately to from 500 to 600° C., mixed and reacted in a special burner with flame formation. The O₂/CH₄ ratio is set to approximately 1:2 so that only incomplete combustion can take place. Both the exothermal oxidation of some of the CH₄ and the endothermal dehydrodimerization of the CH₄ to give acetylene and hydrogen take place in the flame. After a residence time of a few milliseconds, the reaction gas is quenched by spraying in water or quenching oil. About 5 kg of soot are formed per 100 kg of acetylene. The acetylene is usually separated off using an extractant, such as N-methylpyrrolidone or dimethylformarnide. The proportion of acetylene by volume in the cracking gas is about 8%, while the principal components are hydrogen at 57% by volume and carbon monoxide at 26% by volume, which in this ratio represent a highly suitable synthesis gas.

The temperatures in this process are, as in other known acetylene preparation methods, above 1400° C. In order to prevent subsequent reactions, the residence times are in the millisecond range, and the reaction gas must then be quenched rapidly by direct injection of a quenchant. During this operation, the corresponding mixture cools to a varying extent, depending on the quenchant employed, and the acetylene is washed out of the resultant mixture by selective solvents.

A refinement of the acetylene/synthesis gas preparation principle described above is disclosed in DE-A 44 22 815 by the applicant. The starting mixture here is generated in a conventional manner in a mixing chamber after separate prewarming. The combustion with acetylene formation takes place subsequently at a burner block arranged in a burner chamber. The channels of the burner block are covered on the exit side by a perforated plate. This allows the relative proportion between the acetylene and synthesis gas in the acetylene/synthesis gas mixture obtained to be varied within broad limits.

A disadvantage of the Sachsse-Bartholomé process is that, in order to achieve a high acetylene concentration in the cracking gas, the hydrocarbons employed as starting material and the oxygen are prewarmed to temperatures between about 300 and 700° C. and then mixed. An ignitable mixture is produced in this way at an oxygen/hydrocarbon ratio of about 1:2. Premature combustion of the hydrocarbon can only be avoided by special design measures, by means of which the residence time in the mixing zone upstream of the burner block is kept short. However, there is always a certain uncertainty and risk potential in the handling of an ignitable mixture. If the process parameters exceed or fall below certain limit values, but also due to faults originating from the plant material, such as ingress of rust particles into the gas mixture, unpredictable ignition and reductions in yield may occur.

A further disadvantage is that the quenching results in a significant amount of thermal energy being lost or optimum recovery of the energy becoming impossible. The corresponding quench liquid physically has, after use, maximum temperatures of 300° C., although it arises at a high temperature level of from about 1500 to 1600° C. The area of application of such a relatively cool liquid for heating purposes is greatly restricted. The high energy consumption necessary for the generation of the reaction temperature above 1400° C. should ultimately likewise be mentioned as a further disadvantage like the significant soot formation, which is particularly pronounced at the high process temperatures. The soot formation not only reduces the yield of synthesis gas and acetylene, but cleaning of the quench liquid soiled by soot or soot coke and of the cracking-gas mixture is also complex.

In order to circumvent the disadvantages outlined above, DE-A 199 14 226.2 by the applicant discloses a low-temperature process which is characterized by a process structure which is different in principle from that known hitherto and is suitable for the preparation of acetylene/synthesis gas mixtures by partial combustion of hydrocarbons. This process for the separation of acetylene and synthesis gas by thermal treatment of a suitable hydrocarbon in the presence of oxygen is, in contrast to the processes disclosed hitherto, carried out at maximum temperatures of 1400° C.

The starting mixture for the preparation of acetylene and synthesis gas can be heated, for example, by ignition, by the supply of energy or by exothermic reactions with simultaneous or prior supply of energy. The starting mixture or the reaction mixtures forming from the starting mixture accordingly have, in accordance with the invention, maximum temperatures of 1400° C. during the process.

In contrast to known processes, the mean residence time in the reactor is comparatively long and is generally at least 10 ms; in a preferred embodiment, cooling is effected by indirect cooling.

The thermal treatment is generally carried out at temperatures of from 1200° C. to 1400° C. The range up to 1350° C. is particularly preferred.

The advantages of the lower process temperature are firstly the reduced soot formation and secondly, the energy demand is significantly lower than in the processes hitherto, and effective energy recovery is possible.

Oxygen is usually supplied to the starting mixture in the form of air, oxygen or air/oxygen mixtures. It is also possible to add steam and/or carbon dioxide as oxygen source. The addition of oxygen or air/oxygen mixtures is preferred. These are added to the reaction mixture in such an amount that the oxygen/carbon molar ratio is from 0.1 to 0.8, preferably from 0.4 to 0.6

The composition of the starting mixture depends on the use of the cracking gas to be prepared. Important cracking gases which can be prepared by means of the outlined process are, for example, acetylene/methanol synthesis gas, acetylene/ammonia synthesis gas, acetylene/hydrogen rich gas, acetylene/carbon monoxide rich gas, acetylene/oxo gas and acetylene/ethylene synthesis gas. Besides natural gas, liquid gas (propane or butane), petroleum ether, aromatics, pyrolysis benzine oil (from cracking processes) and/or vacuum distillation residues from petroleum refineries can be used. The starting mixtures may contain up to about 10% by volume of recycled cracking gas, recycle gases, such as residual methane from cracking-gas cleaning, residual gases from other processes or synthesis gas. Oxygen can be added to the starting mixture in the form of air, giving ammonia synthesis gas and acetylene. Addition of steam to natural gas having a high methane content preferentially gives acetylene/hydrogen rich gas, and the use of higher hydrocarbons gives an acetylene/carbon monoxide rich gas.

The contents of the application DE-A 199 14 226.2 relating to the process for the preparation of acetylene/synthesis gas mixtures by combustion of hydrocarbons in the presence of oxygen and/or oxygen-containing compounds are an important and integral part of the present application and are incorporated into the present application by way of reference.

The process disclosed in DE-A 199 14 226.2, which is carried out with various compositions of the starting materials and at various pressures, temperatures and residence times, depending on the desired composition of the acetylene/synthesis gas mixture, should also, if desired, be carried out in such a way that ignitable mixtures of the reactants employed do not occur before the reactant mixture is oxidized at the burner with flame formation.

It is an object of the present invention to provide a burner block by means of which the low-temperature process for the preparation of acetylene/synthesis gas mixtures can be carried out and the presence of these ignitable mixtures in this process can be avoided.

We have found that this object is achieved by a burner block which has a base containing a plurality of channels which are intended for the passage of carbon/oxygen mixtures. Also present is an oxygen channel, through which air, oxygen or an air/oxygen mixture exits.

The combustion of the resultant hydrocarbon/oxygen mixture commences after flow through the burner block.

Oxygen is supplied to the system in the form of air, oxygen or oxygen/air mixtures. It is also possible to add steam or carbon dioxide as oxygen source. This applies both to the hydrocarbon/oxygen mixture and to the oxygen entering through the oxygen channel. All the above-mentioned compounds which are suitable as oxygen source are referred to below as oxygen.

The burner block according to the invention consists of a base containing a plurality of hydrocarbon channels. The hydrocarbon used as starting material, which may be, for example, methane, natural gas, liquid gas (propane/butane), petroleum ether, an aromatic mixture, pyrolysis benzine or even residue oil, passes through these channels. The starting material has been mixed in advance with air, oxygen, an air/oxygen mixture or an oxygen-containing compound, for example water or carbon dioxide. In contrast to, for example, the Sachsse-Bartholomé process, the burner block according to the invention firstly allows addition to the hydrocarbon of an amount of oxygen with which an ignitable mixture does not form and nevertheless stable ignition and combustion in the subsequent reaction space are achieved. The carbon/oxygen mixture can be prepared without particular precautions and can pass through the mixing block and the parts upstream of the burner block with comparatively long residence times.

This is achieved through the burner block containing at least one oxygen channel via which further oxygen is added to the hydrocarbon/oxygen mixture prepared in advance and exiting through the channels. This oxygen channel is preferably located centrally within a plurality of hydrocarbon channels. In particular, this oxygen channel is arranged concentrically within a plurality of hydrocarbon channels. If a plurality of oxygen channels is present, the arrangement described can be repeated as often as desired. It is also possible to combine a plurality of individual burner blocks having the oxygen channel/hydrocarbon channel arrangement described to give a single, coherent burner block.

The composition of the non-ignitable hydrocarbon/oxygen mixture prepared upstream of the burner block naturally depends on the temperature of this mixture after mixing, before entering the burner block. The ignition limits of corresponding starting mixtures are known to the person skilled in the art or are revealed by corresponding tables or can be determined experimentally. It is preferred for the hydrocarbon/oxygen mixture to be prepared and allowed to enter the burner block at room temperature. In this case, on use of natural gas, mixtures having a methane content of >60% by volume are used if pure oxygen is used.

In a preferred embodiment of the present invention, an ignition burner is furthermore installed in the burner block according to the invention. This has the purpose of igniting the mixture of hydrocarbon and oxygen forming after passage through the burner block. An ignition burner of this type is basically only necessary for igniting the mixture at the beginning of the reaction. If desired, the ignition burner can also serve to ensure ignition stabilization of the burning mixture. The ignition burner is preferably installed in the region between the hydrocarbon channels and oxygen channels.

The burner block according to the invention guarantees a range of process-engineering advantages. Thus, the hydrocarbon/oxygen mixture can be mixed in a relatively simple manner giving only coarse mixing of the reactants. In addition, it is not necessary to prewarm the reactants to elevated temperatures. It is entirely sufficient to premix the reactants at room temperature. In addition to the favorable energy balance, the performance of the mixing operation at room temperature has the advantage that a mixture can be prepared which has a high oxygen content, but which is not ignitable and can be handled without special measures. Only after passing through the burner channel, mixing with further oxygen and heating by the temperatures prevailing at the burner block does an ignitable mixture form. The mixtures formed in this way are ideal for the preparation of acetylene/synthesis gas mixtures at temperatures of <1400° C. At the same time, the burner has a large control range regarding the throughput and stoichiometry of the mixture.

The burner block according to the invention is suitable for the preparation of acetylene/synthesis gas mixtures from hydrocarbons. Thus, for example, acetylene/methanol synthesis gas, acetylene/ammonia synthesis gas, acetylene/hydrogen rich gas, acetylene/carbon monoxide rich gas, acetylene/oxo gas and acetylene/ethylene synthesis gas can be prepared. The reaction is carried put at any desired pressure, preferably atmospheric pressure. Suitable reactors are preferably flame reactors or tubular reactors, even without formation of a flame zone.

The burner block according to the invention can also be used for the production of synthesis gas mixtures with no production of acetylene. The hydrocarbons described above can then be used as starting product. These are mixed with oxygen in the desired ratio and then added to the burner block according to the invention. The combustion is then carried out under the usual conditions (temperature, pressure, residence time) for the production of synthesis gas, under which the acetylene yield disappears.

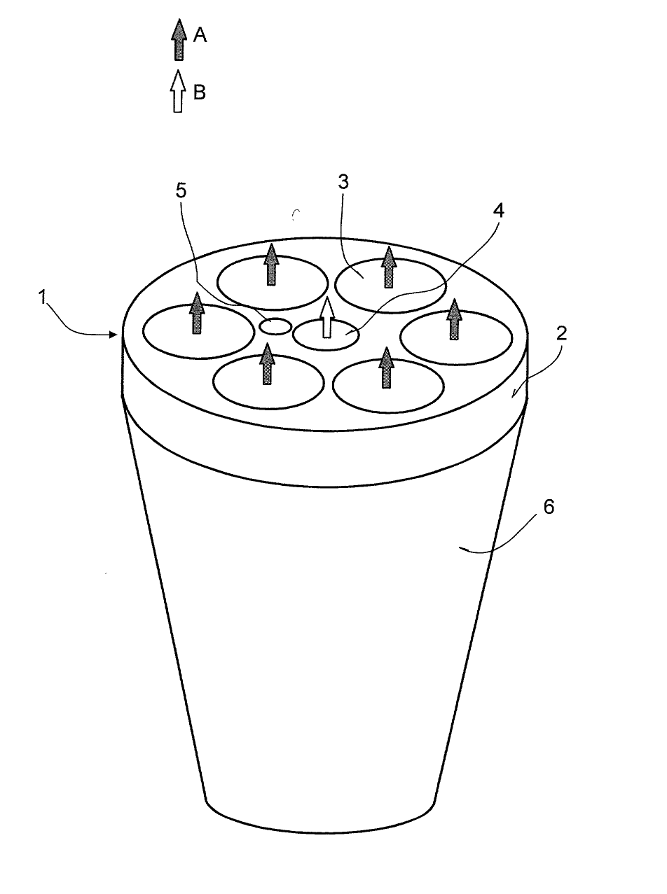

The attached FIG. 1 shows by way of example a burner block 1 according to the invention in a preferred embodiment. This consists of a burner base 2, containing hydrocarbon channels 3. The hydrocarbon channels are arranged concentrically around an oxygen channel 4. The hydrocarbon premixed in advance with oxygen to give a mixture which is not ignitable at the mixing temperature, flows through the burner block 1 through the channel 3 and is then mixed with oxygen after exit. Intensive mixing results, the mixture then ignites and burns to give the desired gas mixture at temperatures of <1400° C. An ignition burner 5 ensures ignition of the mixture. The mixing of the hydrocarbon with the oxygen preferably takes place in a mixing chamber 6 upstream of the burner block.

A hydrocarbon/oxygen exit

B oxygen and/or air/oxygen exit

### EXAMPLE

In a burner block as shown in FIG. 1, 200 m³(s.t.p.)/h of natural gas are mixed via the mixing chamber with 90 m³(s.t.p.)/h of oxygen, and the mixture exits through the hydrocarbon channels. 30 m³(s.t.p.)/h of oxygen flow out through the central oxygen channel. After ignition by the ignition burner, a flame stabilizes downstream of the burner. The natural gas/oxygen mixture is converted under these oxygen deficiency conditions by the flame and the subsequent post-reaction zone at a mean temperature of 1300° C. into a mixture comprising 6% by volume of acetylene, 7% by volume of methane, 20% by volume of carbon monoxide, 40% by volume of hydrogen, 3% by volume of carbon dioxide, the remainder steam and small amounts of soot and higher hydrocarbons.

We claim:

1. A burner block (1) having a base (2), a plurality of hydrocarbon channels (3) for passage of hydrocarbon/oxygen mixtures used as reactants, and at least one oxygen channel (4) for the passage of air, oxygen or an air/oxygen mixture.

2. A burner block as claimed in claim 1, wherein the oxygen channel (4) is surrounded by a plurality of hydrocarbon channels (3).

3. A burner block as claimed in claim 1, wherein the oxygen channel (4) is surrounded concentrically by hydrocarbon channels (3).

4. A burner block as claimed in claim 1, wherein an ignition burner (5) is present.

5. A burner block as claimed in one of claim 1, wherein a mixing chamber (6) is present upstream of the burner block (1).

6. The method of the preparation of acetylene/synthesis gas mixtures from hydrocarbons and oxygen at temperatures below 1400° C., wherein a burner block as claimed in claim 1 is used.

7. The method as claimed in claim 6, wherein the acetylene/synthesis gas mixture is selected from the group consisting of acetylene/methanol synthesis gas, acetylene/ammonia synthesis gas, acetylene/hydrogen rich gas, acetylene/carbon monoxide rich gas, acetylene/oxo gas and acetylene/ethylene synthesis gas.

8. The method as claimed in claim 6, wherein a hydrocarbon/oxygen mixture which is not ignitable at the prevailing temperature enters the burner block.

9. The method as claimed in claims 6, wherein the hydrocarbon employed is methane, natural gas, liquid gas, petroleum ether, an aromatic mixture, pyrolysis benzine or residue oil.

10. The method as claimed in claim 8, wherein the hydrocarbon/oxygen mixture enters the burner block at room temperature and comprises >60% by volume of methane.

11. The method of the preparation of synthesis gas mixtures from hydrocarbons and oxygen under conditions under which the acetylene yield disappears, wherein a burner block as claimed in claim 1 is used.
